(19) United States
(12) Patent Application Publication
(10) Pub. No.: US 2026/0008078 A1
(43) Pub. Date: Jan. 8, 2026

(54) RAPID MERCURY-FREE PHOTOCHEMICAL MICROENCAPSULATION/NANOENCAPSULATION AT AMBIENT CONDITIONS

(71) Applicants: University of Doha for Science and Technology, Doha (QA); AUCKLAND UNISERVICES LIMITED, Auckland (NZ)

(72) Inventors: Jamal KURDI, Doha (QA); Mohammed FARID, Auckland (NZ)

(21) Appl. No.: 19/034,042

(22) Filed: Jan. 22, 2025

Related U.S. Application Data
(63) Continuation-in-part of application No. 18/136,106, filed on Apr. 18, 2023, now Pat. No. 12,233,434.
Application No. 18/136,106 is a division of application No. 17/714,874, filed on Apr. 6, 2022, now Pat. No. 11,666,940.
Application No. 17/714,874 is a continuation of application No. PCT/IB2020/059540, filed on Oct. 11, 2020.
(60) Provisional application No. 62/914,149, filed on Oct. 11, 2019.

(51) Int. Cl. B05D 3/06; A61K 9/50; B01J 13/16; B82Y 40/00

(52) U.S. Cl. CPC B05D 3/067; A61K 9/5089; B01J 13/16; B82Y 40/00

(57) ABSTRACT

Described herein is a method of mercury-free photochemical micro-/nano-encapsulation of an active material for obtaining high-quality shell-core micro-/nano-capsule by means of photo-reaction by UV LED radiation at ambient or even cold temperatures. The method uses appropriate formulation and proper processing steps using a stirrer photo-reactor made from glass or transparent plastics but mixed flow LED-reactor could be also employed. Appropriate gentle mixing is sufficient to expose all droplets, which contain the active material surrounded by curable-shell. Using the optimum light intensities and reactions' times is critical for encapsulating the active material with a high efficiency and producing a high quality micro-/nano-capsules. Light emitted diode (LED) is a mercury-free UV radiation source with a long operating life time and an instant ON-Off. it has a high efficiency, a very low cooling requirements and cost-efficient in photochemical encapsulation. The use of a rapid dissociation triplet Norrish-Type I free-radical photo-initiator with a complete photobleaching ability and good solubility in the curable resins leads to a reduction in time encapsulation from 6 hours to a less than 5 minutes. The formulation of an active substance, photo-curable resins, and miscible initiator or initiators in an immiscible light-transparent continuous liquid phase is emulsified and highly stabilized by the utilization of a single bifunctional stabilizer-emulsifier molecule, which eliminates the need for surfactants. Avoiding the use of surfactant allows for easy separation of the generated capsules from the liquid phase. Consequently, in comparison to alternative techniques, this approach lowers the cost of microencapsulation by minimizing waste water treatment and reducing the loss of unconverted monomers and residual active phase change material (PCM). Additionally, only a specific range of suitable LED radiation is chosen, excluding unsuitable wavelengths, eliminating the generation of heat, which lowers the quality of the finished capsules. Under ideal circumstances, encapsulation efficiency can reach 100% and more than 90% of monomers can be converted. This is on top of the technology's capacity to encapsulate heat-sensitive and volatile active components at both ambient and low temperatures.

### CROSS REFERENCE TO RELATED APPLICATIONS

This application is a continuation-in-part of U.S. patent application Ser. No. 18/136,106 filed Apr. 18, 2023, which is a divisional application of U.S. patent application Ser. No. 17/714,874, filed Apr. 6, 2022, which is a filing under 35 U.S.C. § 371 of International Application No. PCT/IB2020/059540, filed Oct. 11, 2020, which claims the benefit of U.S. Provisional Application No. 62/914,149, filed Oct. 11, 2019. The entirety of each of these applications is herein incorporated by reference.

### BACKGROUND OF THE INVENTION

### 1. FIELD OF THE INVENTION

The present invention relates to mercury-free photochemical micro-/nano-encapsulation, and particularly to a method for a rapid photochemical encapsulation (at micro or nano scale) of an active material at ambient conditions/low temperature, that utilizes optimum formulation, narrow/single wavelength radiation (such as Light Emitted Diode (LED) light or solar monochromator device) at optimum light intensities and reactions' times in an appropriate stirrer or mixed flow photoreactor.

### 2. DESCRIPTION OF THE RELATED ART

Microencapsulation and nanoencapsulation are used to totally or partially isolate from the surrounding environment any active materials in a wide range of industrial applications such as energy storage using phase change materials, food, household, chemicals, additives, agrochemicals, textiles, printing, petroleum, cosmetic, medical, pharmaceutical and others [1-4]. The coated shell layer can be tailored to be a stimulus-responsive polymer or to have the ability in totally sealing or controlling the release of an active material in an ideal place at the required times [5]. The purpose of encapsulation is for obtaining the most desired characteristics and efficient usage of the active material in a given application [6].

Microencapsulation techniques can be classified into three main categories: (1) Physical/mechanical methods where coating materials are formed from polymers through physical processes (such as solvent evaporation, melt solidification, . . . etc.); (2) physico-chemical methods such as sol-gel and coacervation encapsulation; and (3) chemical methods in which the coating materials are formed from monomers or oligomers through chemical reactions such as polymerization, poly-condensation, chemical curing and crosslinking. Each technique produces different microcapsules as reported elsewhere [7-9]. From these, chemical microencapsulation such as interfacial and in-situ polymerization, poly-condensation, curing and crosslinking have gained more industrial attention compared to other techniques because it produces more stable and durable microcapsules [10].

In the current state of the art, physical and chemical encapsulation requires a high temperature for melting, spray drying, polymerizing, curing or crosslinking the coating materials. This will cause the following challenges:
1. Thermal microencapsulation requires a costly long reaction time (2 to 6 hours) at a high temperature (50° C. to 80° C.) and hence high energy.
2. Interfacial microencapsulation requires a costly long reaction time (2 hours at 35° C.) or a relatively high costly heating (15 minutes at 70° C.) or for one hour at 40° C. as

reported in U.S. Pat. No. 5,164,126 that disclosed adding a step after the emulsion step and before starting encapsulation reaction, which is delaying polyamine addition for two hours at elevated temperatures. This step increases the processing costs significantly, and may override other benefits. Encapsulation yield and reaction efficiency were not determined in their reported patent.
3. The inability for coating large number of thermo-sensitive active materials (including biomaterials) since high temperature may cause volatilization and/or destruction of these materials [5].
4. For thermal encapsulation of PCM, the highest reported heat of fusion is 113.4 J/g when (Rubitherm® RT21). The encapsulation requires 6 hours at 80° C. to achieve 86.47% monomer conversion [13].

To overcome the above challenges, the invention described herein targets micro-/nano-encapsulation of 100% of the active material by using photo-encapsulation temperature of 22° C. for 5 minutes or less.

Chemical microencapsulation can be achieved as disclosed herein at ambient conditions (T =22° C.) or even colder temperatures by using UV photo-initiators instead of the thermal initiators for micro-/nano-encapsulation of an active core material. Microencapsulation at a low temperature and methods of using a photoreactor for microencapsulation of an active material such as phase change materials or other active materials is described in U.S. Pat. No. 10,913,882. The idea of using photo microencapsulation at ambient or low temperatures is also described in WO/2017/040699A1, US 2018/0223146 A1, EP3344381, EP3344381A4, EP3344381A1, EP3733276A1, and U.S. Pat. No. 10,913,882B2.

Prodo Laboratories, Inc. described photo encapsulation at a temperature of 37° C. in PCT Application WO 2017/151773 wherein polymerizable high density ethylenically unsaturated polyethylene glycol (PEG) having a molecular weight between 900 and 20,000 Daltons, and a sulfonated comonomer for the photo encapsulating devices or cell aggregates by micro-bulk capsules or coating is claimed. In the method disclosed by Prodo Laboratories, Inc., emulsion encapsulation is implemented inside the continuous phase using a water-soluble expensive reactive macromers. A 5% concentration of 10 kDa PEG tetra-acrylate is required to form acceptable micro-bulk capsules using 20 second radiation exposure time. The used hydrophobic material is just an inert template to form the coating gel shell that contains active bio-material. It is difficult to form a superior sealing shell layer with a core of an active material by this method. This is because both the active biomaterial and macromers are hydrophilic in nature. Also, the crosslinking within a short time is attributed to the high molecular weight of the macromer and the presence of four reactive cross-linkable functional groups in each macromer. The active living biomaterial is glued or embedded within the coating layer, and there is no need for sealing because oxygen should diffuse from air to the active biomaterial. Economically, the encapsulation methodology disclosed by Prado cannot be feasible for most other widespread applications due to the fact that using PEG-tetraacrylate macromer has a low encapsulation efficiency. The reaction is restricted only to the near surface and not the entire macromer solution is polymerized or gelled, because diffusion is a much slower process than polymerization. This challenge was reported in U.S. Pat. Nos. 6,258,870 and 5,573,934. This is in addition to the high cost of the macromers.

The price of 1 gram of commercial 4arm-PEG10K-Acrylate average MW 10,000 is US$246.50 [15], while the price of 1 Kg of the methyl methacrylate monomer is US$1.9 [16]. The method described herein is different in terms of economic feasibility as well as producing desired superior quality of the shell-core capsules required in most other commercial widespread applications. Therefore, using the highly expensive macromers is not recommended.

The herein invention is about the use of novel emulsion stabilizer without surfactant for confining the dispersed phase at a desired micro-/nano scale. Design, assemble and test of a new LED photo-reactor for photo-encapsulation, by which all problems with the existing photoreactors used in emulsion photopolymerization can be solved by using a very rapid dissociation triplet Norrish-Type I free-radical photo-initiator with a complete photo-bleaching ability and LED with adjustable light intensity which has a narrow/single wavelength radiation for a cost-efficient micro-/nano-encapsulation of active materials within a micro-/nano-scale space. All materials and encapsulation processes occur in the confined space. The role of the continuous phase is only to carry and rotate the shell-core capsules discretely before and after the photo-encapsulation process. In addition to dissipative heat generated by the exothermic photo-encapsulation.

### 3. SUMMARY OF THE INVENTIONS

Described herein is a method of mercury-free photochemical micro-/nano-encapsulation of an active material for obtaining high-quality shell-core micro-/nano-capsule by means of photo-reaction by UV LED radiation at ambient or even cold temperatures. The method uses appropriate formulation and proper processing steps using a stirrer photo-reactor made from glass or transparent plastics, but mixed flow LED-reactor could be also employed. Appropriate gentle mixing is sufficient to expose all droplets, which contain the active material surrounded by curable-shell. Using the optimum light intensities and reactions' times is critical for encapsulating the active material with a high efficiency and producing a high quality micro-/nano-capsules. Light emitted diode (LED) is a mercury-free UV radiation source with a long operating life time and an instant ON-Off. it has a high efficiency, a very low cooling requirements and cost-efficient in photochemical encapsulation. The use of a rapid dissociation triplet Norrish-Type I free-radical photo-initiator with a complete photobleaching ability and good solubility in the curable resins leads to a reduction in time encapsulation from 6 hours to a less than 5 minutes. The formulation of an active substance, photo-curable resins, and miscible initiator or initiators in an immiscible light-transparent continuous liquid phase is emulsified and highly stabilized by the utilization of a single bifunctional stabilizer-emulsifier molecule, which eliminates the need for surfactants. Avoiding the use of surfactant allows for easy separation of the generated capsules from the liquid phase. Consequently, in comparison to alternative techniques, this approach lowers the cost of microencapsulation by minimizing waste water treatment and reducing the loss of unconverted monomers and residual active phase change material (PCM). Additionally, only a specific range of suitable LED radiation is chosen, excluding unsuitable wavelengths, eliminating the generation of heat, which lowers the quality of the finished capsules. Under ideal circumstances, encapsulation efficiency can reach 100% and more than 90% of monomers can be converted. This is on top of the technology's capacity to encapsulate heat-sensitive and volatile active components at both ambient and low temperatures.

The present invention relates to an eco-cost-efficient method for a rapid photochemical micro/nano-encapsulation of an active material with a polymeric shell at an ambient or colder temperature, using a variety of improved scalable processes. For polymeric micro/nano-encapsulation, the use of a modified bi-functional electro-steric stabilizer efficiently for dispersing, confining and stabilizing both the hydrophobic active material and the photo-curable resins (including at least one photo-initiator and all optional additives) in a mainly immiscible low-light-scattering/absorbing continuous liquid phase is disclosed. For hydrophilic active materials, the use of a polyol-hydrophobic bi-functional stabilizer instead of the electro-steric polymer is described. The emulsion is formed by using a high shear homogenizer which is well-known in the state of art. Then, a very rapid dissociation triplet photo-initiator with a high photo-reactivity is used for the formation of a perfect shell of the capsules through a rapid and a high yield photo polymerization of the curable resins.

An appropriate semi-batch/continuous flow tubular photo-reactor equipped with adjustable intensity of a collimated narrow/single wavelength radiation using Light Emitted Diode (LED) light or solar monochromator device is designed. In addition, an appropriate stirring system, which creates a laminar low-shear mixing and a gentle radial movement of the dispersed droplets in the emulsion along the entire length of the tubular reactor, is used. As described in U.S. Pat. No. 10,913,882B2, in certain embodiments, fouling, cleaning, having a high-shear resulting from flow a thin film on large surface area, and forcing circulation of the emulsion using a pump resulted. It was recognized that in certain embodiments, pumping the emulsion and having a large contact surface area between the wall of the thin-film reactor and the emulsion can cause deforming, breaking and coagulating the capsules during the early stages of their formation. This was observed through fouling and the polymerization of monomers on the wall of the reactor instead of polymerization of the monomers inside the dispersed phase.

Other critical problems in existing photoreactors used for encapsulation are light attenuation, non-homogeneous beam profile, and long light paths that go through different materials and have different refractive indexes. Scattering and reflecting diffracting light may occur due to poor design. These problems are less significant for photoreactors used in emulsion polymerization of polymeric particles or curing of small thin-film bulk resins. This is because much higher light intensity (by 10 fold) is required for photo-encapsulation. The new design described herein was developed to overcome these challenges while also acknowledging the limit deep penetration of radiation (1 to 2 cm) into the dispersed droplets inside the emulsion medium.

Based on experimental results described herein, a compact space-effective cylindrical or square tube with a transparent durable rigid wall with a cross-section diameter from 3 to 4 cm. Using a larger diameter leads to a decrease in the space-effectiveness of the reactor. Radiation should be aligned and configured for minimizing the attenuation and non-homogeneous beam profile; the optical path length should be as short as possible with an optimum and appropriate irradiation conditions; the difference in the refractive index between phases should be minimized; and the radiation penetration inside the emulsion has to be increased by decreasing the droplet content and/or diameter. In the design described herein, the LED lamp touches the tube perpendicularly to eliminate the presence of air gap. The LED lamps have a powerful radiation up to 24000 mw/cm² and a wavelength range between 350 nm and 440 nm. It is preferred to select or modify all used materials to have a low similar refractive index. The stirring system inside the reactor should be designed to create radial rotational laminar gentle mixing, which should be sufficient to expose all dispersed droplets from all sides to radiation. The helical rounded-wire impeller along the entire tube at a stirrer speed below 600 rpm is preferred. High shear mixing and whipping that causes the deformation, coagulation, rupture of the dispersed droplets, and/or the formation of gas bubbles and foams should be avoided. The designed reactor is scalable and can be for continuous and semi-batch processes with a uniform-effective heat dissipation. Notably, the source of radiation (such as LED) should have a much higher intensity for encapsulation than the ones used for photopolymerization of the thin-film bulk or polymeric nanoparticle as known in the state of art. Based on using the new photoreactor design compared to that implemented in the previous existing designs, experimental observations show less or no fouling, superior shell-core micro-/nano-capsules and high efficiency processes.

The method described herein includes many novel processes that lead to complete 100% active material encapsulation, a yield above 95%, and with monomeric conversion of above 90% at a photo encapsulation temperature as low as 20° C. for 5 minutes. The novelty of these processes is in producing the desired superior shell-core micro/nanocapsules at ambient conditions. Further as disclosed herein, this process greatly minimizes cost through reducing waste of chemicals; increases encapsulation and photoreactor efficiencies; greatly reduces utilities costs by processing at ambient conditions; and uses for the first time a confined strongly stabilized dispersed phase with a highly efficient photopolymerization method inside micron-/nano-compatible space.

Described herein is a novel process for the formation of a highly stable emulsion. The process is based on using one modified electro-steric stabilizer, which can spread uniformly around the dispersed phase and form a steric shell layer sufficient for complete confinement of the dispersed droplets. The selected stabilization layer (such as sulfonated polyvinylalcohols) is suitable for photopolymerization because it has a high transparency greater than 95% for radiations over 350 nm wavelengths and a refractive index of 1.50 in the visible electromagnetic radiations. This is in addition to the ability to reduce the differences in refractive indices and the reflection loss with increasing radiation output. The high stability of the emulsion in the photo-reactor will continue until an underneath new layer of polymeric shell is formed by photo-bleach polymerizing, crosslinking and/or curing all monomers/oligomers resins. In the existing state of art, stabilization of the dispersed phase is achieved by using a combination of electrostatic surfactant and steric polymeric stabilizer. The presence of the hydrophobic tails in the ionic surfactant causes a problem that negatively affects the formation of the superior stabilization layer as discussed herein. Experimental results described herein indicate and confirm that chemical binding an ionic functional group to the steric polymeric stabilizer (instead of adding ionic surfactant to the mixture) can produce much superior stabilization and a confinement layer surrounding the dispersed droplets. The ionic functional group has a high affinity to the water continuous phase, while the steric part of the polymer is oriented towards the inside of the dispersed phase. The steric polymer layer can be tailored to have a high affinity to the curable resins and can be suitable to create a confined micron/nano-space for a rapid free-radical polymerization. In reverse emulsion where the active material is highly hydrophilic in nature, the preferred stabilizer is a modified polyol with chemically attached hydrophobic moieties. As preferred examples, the polyglycerol esters of fatty acids can be used for formation of an efficient water-oil stabilizer.

Further, a very rapid dissociation triplet Norrish-Type I free-radical photo-initiator with a complete photo-bleaching ability can be used effectively as a photinitiator within an appropriate stable steric confined micron/nano-space. The highly stable transparent steric colloidal-protective layer works as an appropriate environment and as a micro-/nano photoreactor in which the alpha-cleavage (type I) photoinitiator and the resins (containing at least one monomer and one cross-linking agent) with an active material form superior shell-core capsules through a rapid and a high yield efficient photo polymerization process at ambient or below ambient temperatures. Experiments described herein confirmed that the photopolymerization and encapsulation processes are not affected by the presence of the small amount of inhibitors already existing in the commercial monomers. Also, oxygen does not influence the inhibition of the selected free-radical photo-initiator.

Our novel process for polymeric encapsulation is based on photopolymerizing, crosslinking, and/or curing inside a micro/nano-scale confined space. Monomers/oligomers and other components do not need to diffuse to the interface or form large micelle droplets to nano-micelle droplets as in the case of emulsion polymerization. The formed electro-steric bilayer stabilizer, which confines the micro-/nano space, works to isolate the entire components used in the encapsulation process including the active core material. This limits oxygen diffusion into the reaction during encapsulation. Therefore, there is no need to purge with an inert gas. Also, there is no need to eliminate and remove entrapped and dissolved gas that can cause light scattering and attenuation. As an ideal theoretical method, the monomers and crosslinking agents should be selected to have a close hydrophilicity to that of active material. The inner side of the stabilization steric layer should have an appropriate hydrophilicity that matches the hydrophilicity of the monomers. The photoinitiator should be soluble in the monomers. The continuous emulsion phase should have an opposite hydrophilicity. The outer side of the stabilizer should have a high affinity to the continuous phase. The entire electro-steric bilayer stabilizer should be transparent to radiations and be flexible to accommodate the shrinkage of the capsule after polymerization of the permanent shell which encapsulates the active material. It is clear from the experiment described herein that the uniform distribution, solubility and the appropriate chemical and physical surrounding environment enhance the reactivity of both curable monomers and photoiniator within a confined micro-/nano space for a highly efficient and rapid encapsulation process. The method is suitable for all active materials as long as the hydrophilicity of all components and the conditions as described herein are met.

The method of eco-cost-efficient polymeric photo-encapsulation of an active material at ambient and low temperatures is herein disclosed. This method is based on the dispersion and confinement of highly stabilized micro-/nano-droplets of photo curable resins which contain the active material needed to be encapsulated for a given application. High shear rotor/stator mixers at a high speed ranging from 4000 rpm to 10000 rpm can be used to form the emulsion as known in the state of art. Photo-polymerizing, crosslinking and curing the selected resins are implemented based on using appropriate formulation and quality-efficient photo-encapsulation setting. Different from designs of the existing reactor settings used in the state of art for photo emulsion polymerization and encapsulation, a new reactor setting appropriate to produce high quality superior shell-core micro-/nano-capsules cost-efficiently was designed and fabricated. The novelty of this new photo reactor design is the ability to implement the photo encapsulation process efficiently without deforming, breaking and coagulating of the dispersed droplets. The stirring system in the new design has the ability to suspend and rotate the stabilized dispersed droplets radially without hitting the wall of the tubular reactor in a low-shear laminar mixing. The droplet should also rotate around itself to expose its entire outer surface from all sides to radiation. The impeller should not have blades with a high contact surface area that beat the emulsion. Pumping the emulsion to flow through the reactor should not be used, but this is replaced by gravity falling flow. The diameter of the transparent tube is preferred to be typically between 3 to 4 cm. Not all dispersed droplets are exposed to radiation at the same time; only droplets close to the reactor wall are exposed to radiation while droplets in the middle of the tube are in dark. With the formation of more cross-linked polymer, the emulsion become more opaque, and a larger number of droplets become dark at one time. At the final stages of photo-encapsulation, a higher light intensity is required to overcome the more opaque emulsion and the slower process at higher monomer conversions. For one droplet, light exposure is not continuous but on and off as the droplet moves between dark space and lighted space. Because this is good for given resins and types of photo-initiators, but not may be not good for other formulations, a larger reactor diameter is preferred when shorter light exposure and longer dark periods are required.

Another aspect of the new photoreactor design is the transmission of appropriate radiation to the dispersed droplets in the emulsion. In micro-/nano-encapsulation processes, light received by resins during photo-reactions is different from the emitted light from the source due to the high heterogeneous of the system compared to more homogeneous system for traditional emulsion polymerization setting. The LED radiations setting in the reactor design described herein has the shortest light transmission path. The LED lamps touch the outer surface of the reactor wall and a high power light source with adjustable intensity between 0 and 24000 mW/cm². Light path is perpendicular to the wall surface. If possible, diffractive index for all materials should be similar and as low as possible. Uniform distribution of LED lamps is required and using light reflective surfaces such as mirrors or any alignment may cause light scattering and should be avoided.

In contrast to the traditional emulsion polymerization, complete monomer conversion is essential to produce high quality and superior shell-core capsules used in most industrial applications. Remaining monomers within the matrix of the shell layer lead to a poor structure and low quality micro-/nano-capsules. Leaching or removing the monomers later ruins the structure of the shell layer. In the current state of art, it is very difficult to reach a complete monomer conversion. As described herein, by applying the three improvement approaches below, high quality micro-/nano capsules were produced in 5 minutes or less encapsulation time at ambient conditions. (1) Using a novel stabilizer to form a temporary protective layer can confine the dispersed droplet. The inner surface of this protective layer can play a role in enhancing the photoreactions, and thus improve monomer conversion particularly when conversion is high but not completed. (2) Using the photoreactor design disclosed herein with appropriate novel stirring system also leads to improved monomer conversion, but does not reach complete monomer conversion. (3) Additional significant improvement has been disclosed herein by using a rapid dissociation triplet Norrish-Type I free-radical photo-initiator with a complete photobleaching ability. This photo-initiator leads to a highly significant increase in the monomer conversion.

### BRIEF DESCRIPTION OF THE DRAWINGS

FIG. 1. Photochemical falling flow stirrer reactor for micro-and nano-encapsulation. A schematic representative model is also shown for the stabilization and confinement of the dispersed droplet in the emulsion.

FIG. 2. Heat of Fusion of PCM microcapsules drying at 55° C. for 12 hours and then, after drying at 55° C. for 20 days. Irradiation at 1.2 W/cm² for 4 minutes.

FIG. 3. Photochemical thin-film flow reactor for micro-and nano-encapsulation.

FIG. 4: DSC thermogram of pure PT 20.

FIG. 5A: DSC measurements of microcapsules treated at 450 W UV intensity for 1 minute.

FIG. 5B: DSC measurements of microcapsules treated at 450 W UV intensity for 2 minutes.

FIG. 5C: DSC measurements of microcapsules treated at 450 W UV intensity for 5 minutes.

FIG. 5D: DSC measurements of microcapsules treated at 450 W UV intensity for 10 minutes.

FIG. 6: Thermogravimetric analysis of samples treated at UV intensity of 450 W.

FIG. 7: Weight analysis of PT 20 microcapsules stored at 50° C. over a span of 40 days.

FIG. 8. Influence of radiation intensity on encapsulation efficiency for 2 minutes reaction time at wavelength of 365 nm.

FIG. 9. Influence of irradiation time on encapsulation efficiency without using the mixer in the photoreactor at a wavelength of 365 nm and a radiation intensity of 1.2 W/cm².

FIG. 10. Influence of irradiation time on encapsulation efficiency at a wavelength of 365 nm and a radiation intensity of 12 W/cm² (maximum light intensity of the LED lamps).

FIG. 11. Influence of irradiation time on encapsulation efficiency at a wavelength of 365 nm and a radiation intensity of 0.6 W/cm².

FIG. 12. Absorption spectra of the bis-acylphosphine oxide (BAPO) photoinitiator (PI) in different monomers and in acetonitrile.

FIG. 13. Absorption spectra of the microencapsulation emulsion without photoinitiator (PI) compared to the spectra of the bis-acylphosphine oxide (BAPO) in monomers and crosslinking agents.

FIG. 14. Absorption spectra of the microencapsulation emulsion with and without photoinitiator (PI) compared to the spectra of the monomers and crosslinking agents.

FIG. 15. Absorption spectra of the microencapsulation emulsion with photoinitiator (PI) before curing and after radiation curing for 3 minutes.

### 4. DETAILED DESCRIPTION OF THE PREFERRED EMBODIMENTS

Described herein is a method of mercury-free photochemical micro-/nano-encapsulation of an active material for obtaining high-quality shell-core micro-/nano-capsule by means of photo-reaction by UV LED radiation at ambient or even cold temperatures. The method uses appropriate formulation and proper processing steps. The resins comprise at least one monomer and one crosslinking agent. Optionally more monomers, oligomers, crosslinking agents, and other promoting additives can be used. A very rapid dissociation triplet Norrish-Type I free-radical photo-initiator with a complete photo-bleaching ability can be used. The photo-initiator should be soluble in the resins. For photo-encapsulation processing, a commercial high shear rotor/stator mixer is used to form a highly stable emulsion at a desired micro-/nano-size for the dispersed droplets. A complete coverage and perfect confinement of these droplet was obtained by using a bi-functional ionic-steric polymeric stabilizer. In case of hydrophilic dispersed phase, nonionic hydrophobic-steric polymeric stabilizer can be used. The chemical nature of the surrounding confinement layer is tailored to enhance the photo-encapsulation process within a micro-/nano-confined-space. A falling flow stirrer photo-reactor made from glass or transparent plastics with a narrow diameter between 3 to 4 cm can be used. A low shear mixing with a gentle radial rotation and spinning of the dispersed droplet in the reactor can be used as an efficient process. With this stirring design, deforming, breaking and coagulating the droplet can be avoided, but a mixed flow reactor could be also employed. The LED lamp should touch the outer side of the wall in order to have the shortest light path and eliminate the air gap between the lamp and the encapsulation reactor. LED lamps with adjustable light intensity which have a narrow/single wavelength radiation are used for a cost-efficient micro-/nano-encapsulation of active materials within a micro-/nano-scale space. Using optimum light intensities and reaction times is critical for encapsulating the active material with a high efficiency and producing a high quality micro-/nano-capsules. A solar monochromator device can also be used as long as it generates the same radiation with narrow/single wavelengths as the LED device.

Light emitted diode (LED) is a mercury-free UV radiation source with a long operating life time and an instant ON-Off. It has a high efficiency, a very low cooling requirement and is cost-efficient in photochemical encapsulation. With use of an appropriate novel stabilizer and a proper rapid free-radical photo-initiator, irradiation by LED lamps reduces the time of microencapsulation from 6 hours to a less than 5 minutes. It has a significant decrease in manufacturing cost, waste-water, unconverted monomers, and leftover active phase change material (PCM) compared to other existing methods. Conversion of more than 90% of monomers can be achieved, and encapsulation efficiency can reach 100% at optimum conditions. This is in addition to the ability of this invented technology to encapsulate volatile and heat sensitive active materials at ambient as well as low temperatures. Normal glass or transparent plastics can be used as a reactor material. Only the matched useful wavelength radiation is emitted by LED without having other wavelengths which might have a bad impact on the encapsulation process.

The method for mercury-free photochemical micro-/nano-encapsulation of an active material in dispersed systems comprises many processing steps for obtaining high quality and superior shell-core micro-/nano-capsules at ambient or even cold temperatures. The first step is the selection of the appropriate compatible materials and chemicals suitable for photo-encapsulation in dispersed systems; by which a shell-core encapsulated active material can be produced for a given application. This selection is based on the hydrophilicity of the given active material. For hydrophobic active material, the preferred resins, and so all components inside the dispersed phase should be hydrophobic in nature. The continuous phase should be immiscible hydrophilic in nature and oil-water emulsion type is used. Water is the preferred a continuous phase. Resins in the dispersed phase comprise at least one monomer or oligomer, optionally one or more crosslinking agents, at least one photo-initiator, and optionally other enhancement additives. All resins components should be hydrophobic and the photo-initiator should be soluble in the resins. The stabilizer should be a steric polymer with chemically attached strong ionic functional groups. An example for this type of stabilizer is the commercial GOHSENX™ L-3266, which is a modified PVOH with a sulfonic group (SO₃/Na group) on its side chain.

For hydrophilic active material, the dispersed phase should be hydrophilic and water-oil reverse emulsion is used. The hydrophilicity of all components should be opposite to that mentioned in the paragraph above. The photo-initiator should be soluble in the resins. The stabilizer is a nonionic polymer surfactant. The continuous phase should be immiscible hydrophobic in nature. When the absolute viscosity of the continuous hydrophobic phase exceeds 2 centipoise, a double W-O-W emulsion might be preferred.

The organic phase and aqueous phase are emulsified using high shear rotor/stator mixer. These mixers with interchangeable stator screens are commercially available from companies such as Charles Ross & Son Company, Silverson, Tetra Pak, Fluko, etc. The speed of these mixers is in the range 200 to 11000 rpm. The speed is selected based on the desired dispersed droplet diameter. Increasing the speed leads to reduction of the diameter of the droplet down to 1 μm. Further, sonication is an additional process to reduce the droplets' diameter to the nanoscale.

Photo-encapsulation of an active material is implemented by irradiation of the emulsion (mentioned in the previous paragraph by an LED radiation source. The available commercial LED lamps in the UV range emit light at a wavelength 365 nm, 385 nm, 395 nm or 405 nm. LED lamps in the visible range are available at violet (400 nm to 450 nm), blue (450 nm to 500 nm), green (500nm to 570 nm), and yellow (570nm to 590 nm). The wavelength of the emitted light from the LED is selected based on the maximum light absorption by the photo-initiator. However, the photo-initiator may have a high and significant light absorption within a range of wavelengths. For photo-encapsulation, longer wavelengths can penetrate deeper in the emulsion. The possibility of absorption of light by resins and other existing components in relationship to the light wavelength should be considered if it is significant. The preferred wavelength is where there is a high light absorption by the photo-initiator, low light absorption by all other existing components, and a deeper penetration of radiation inside the emulsion. As the photo-encapsulation proceeds, more monomers are converted to polymers with time. The refractive index and the optical properties of the formed polymer is different from the ones for monomers. Also, the emulsion becomes opaque with the formation of a crosslinked polymer shell. The penetration of light into the emulsion and into a thicker layer of the polymeric shell becomes very limited to convert the remaining low concertation of monomers A wavelength of 365 nm as well as 395 nm were used. The preferred light intensity for photo-encapsulation is 600 mW/cm² that gradually increases to 1500 mW/cm². Irradiation time of 5 minutes might be sufficient to obtain a high-quality shell-core micro-/nano-capsules. It was surprisingly observed that a longer exposure to a high intensity light after reaching the highest monomer conversion leads to damage to the capsules.

Using the optimum light intensities and reactions' times is critical for encapsulating the active material with a high efficiency and producing a high quality micro-/nano-capsules. A solar monochromator device can also be used as long as it generates the same radiation as the LED device. Compared to using a medium pressure mercury lamp or thermal encapsulation, encapsulation using UV-LED is more energy efficient, more environmentally friendly, radiation with undesirable wavelengths can be excluded, and it is also simple to arrange (no purging with nitrogen, no purification for the used technical materials, not using quartz reactor and so on).

A falling flow photoreactor equipped with a low-shear laminar flow stirrer is used. The preferred speed of the stirrer is 300 rpm. The helical rounded-wire impeller along the entire height of the reactor is preferred because this stirrer rotates the dispersed droplet radially and also, makes the droplet rotate around itself. This leads to a quick on-off light exposure with moving the droplets from the dark space in the middle of the tube to lighten space close to the wall of the tube, and vice versa. The preferred diameter of this tubular reactor is between 3 to 4 cm.

The optimum conditions and processing time depend on the formula of the curable materials, photoinitiators, boost/enhance processing additives, stabilizer/emulsifying agents and emulsion preparation method. In most cases, the total period of the encapsulation process is less than 5 minutes. The encapsulation process is implemented at ambient temperature but also can be done at other temperatures particularly at a colder temperature than the room temperature, if there is a need for this.

The active material can be a known organic phase change material, or any active material used in food, household, chemicals, additives, agrochemicals, textiles, printing, paint, petroleum, cosmetic, detergent, medical, pharmaceutical and others.

The droplet stabilizer should be a bi-functional steric polymer with a strong ionic moieties and polyol moieties in order to obtain a highly stable micro-/nano-confinement. As a preferred example of an Oil-Water electro-steric stabilizer, the commercial Gohsenx (modified PVOH) L-3266 can confine the dispersed droplets in the micro/nano space. Importantly using ionic surfactants destroys the confining layer. The hydrophobic moiety in the ionic surfactant is attracted by the hydrophobic dispersed phase and prevents the steric polymer from completely stabilizing and confining the droplet in the oil-water emulsion. Using only steric polymeric stabilizer without surfactant, the interfacial tension will remain high and cause a less effective dispersion and weak confinement of the dispersed droplets. Using only surfactant without steric stabilizer, dispersion occurs due to lower interfacial tension, but stabilizing and confining the dispersed droplet does not occur. In the selected stabilizer and for hydrophilic dispersed phase, the ionic moiety should be replaced with a hydrophobic moiety.

### EXAMPLES

### Example 1: A Stirrer Photoreactor for Microencapsulation of Commercial Rubitherm® RT21

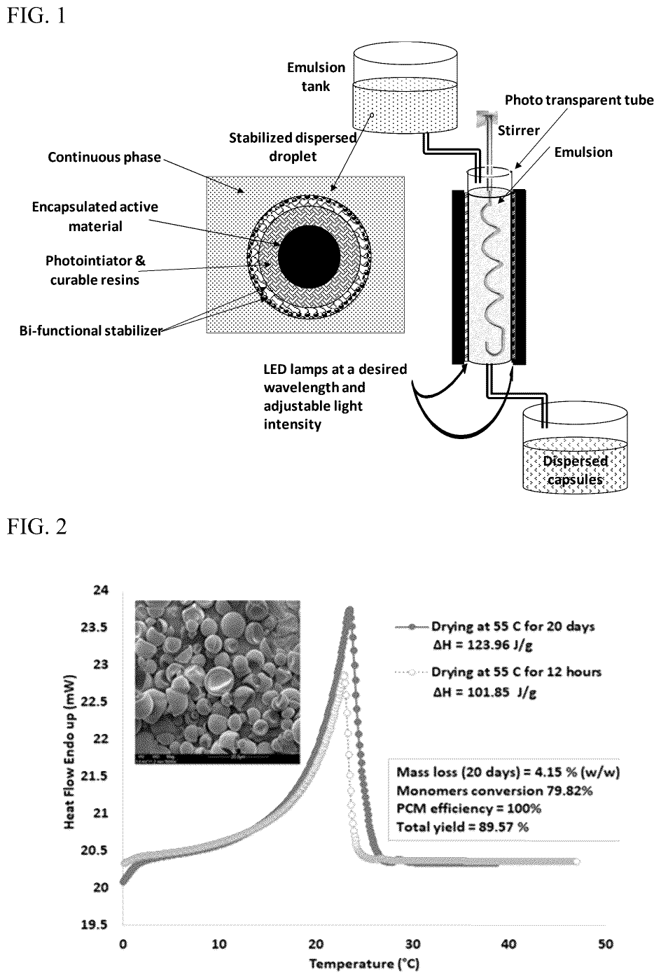

The RT21 microcapsules were prepared in a stirrer photoreactor. As shown in FIG. 1, the LED stirrer photoreactor consisted of a pyrex rounded glass tube with 3.5 cm diameter and 13 cm height. Two identical LED lamps (BETTSENS) at a wavelength of 365 nm and adjustable radiation intensity between 0 and maximum 12 W/cm² contacted the outside of the tube. The light emitted area of each lamp was (11×1.5 cm). The second lamp was turned 90 degree in reference to the first lamp as shown in FIG. 1. A helix stirrer that fits inside the tube was used. In one of the typical experiments, the curable materials consist of a hard-monomer such as MethylMethAcrylate (MMA) or soft-monomer such as Butyl acrylate (BA) or their mixture at a concentration of 70% by weight and 30% by weight of di-tri and/or polyfunctional monomers or oligomers. For a thick layer of curable materials, acylphosphine oxides are very effective photoinitiators. In this example, commercial bis-acylphosphine oxide (Irgacure 819, BAPO) was used at a concentration of 3% by weight of curable materials. The weight ratio of Rubitherm® RT21 to the curable materials is 1. Deionized water was used as a continuous phase in the emulsion with a stabilizer such as the commercial Gohsenx (modified PVOH) L-3266 at a concentration of 1% by weight of the total aqueous phase. This can produce a surfactant-free emulsion. The organic phase and aqueous phase were emulsified using a high shear emulsifier at a speed of 4500 rpm for 30 minutes. The Pyrex glass tube was filled with the emulsion, and the irradiation by the two LED lamps was applied while mixing the emulsion. The produced microcapsules were separated from the suspension by filtration, washed and dried in an atmospheric oven at 55° C. for at least 12 hours. Additional drying for a long time also was applied. Microcapsules drying at different time were tested.

Experiments in Example 1 were conducted at technical conditions without any improvement in order to be compared with the traditional thermal encapsulation. For instance, all curable materials were not purified (i.e., the hydroquinone inhibitor was not removed from the monomers), purging with nitrogen was not done, Pyrex glass tube was used without the need for an expensive quartz tube, enhancement additives were not added, and the weight ratio of the active material (RT21 in this example) to the curable shell materials was not optimized. The results from Example 1 were excellent compared to the thermal encapsulation as shown in FIG. 2. Room temperature was used during encapsulation instead of 80° C. Total encapsulation time was reduced from 6 hours to 4 minutes.

Most of the PCM was encapsulated while in the thermal method, up to 98.5% of PCM was encapsulated in the method of Example 1. The monomer conversion and the total yield of the product was 79.82% (w/w) and 89.57% (w/w), respectively. The thermal method has a higher monomer conversion and total yield by less than 7%. However, the heat of fusion of the dried microcapsules was 123.96 J/g (in Example 1, FIG. 2), which is significantly higher than those commercially available. It is clear from the SEM image in FIG. 2 that most of the microcapsules are deflated as indicated by the axisymmetric dimple formed on one side of the spherical shells. Buckling of the spherical shell of the microcapsules might be attributed to the reduction of the volume inside the capsules during the drying process or because of the collusion among capsules as a result of mixing while encapsulation. However, the increase in the heat of fusion of the PCM-microcapsules upon drying at 55° C. for 20 days indicates that the PCM is well contained. Also, none of the capsules were broken, and there is a possibility that some non-polymerized monomers such as MMA are entrapped inside the capsules with the PCM. Upon drying for long time, MMA is a volatile component that would evaporate outside of the capsules, causing a decrease in the volume of the core materials. Most probably, the PCM to curable materials ratio should be increased in order to optimize the use of the extra non-polymerized monomers. The thickness of the shell curable materials depends on both the ratio of PCM to the curable materials and the size of the microcapsules. In fact, the size of the microcapsules can be controlled by adjusting the speed of the emulsifier and the use of the sonication. It can be concluded that for each desirable capsule size, there is an optimum ratio of the active material to the curable shell material. This optimum ratio is different for different capsules sizes.

### Example 2: Microencapsulation of Puretemp®20 (PT20) Using Thin-Film Flow Photo-Reactor

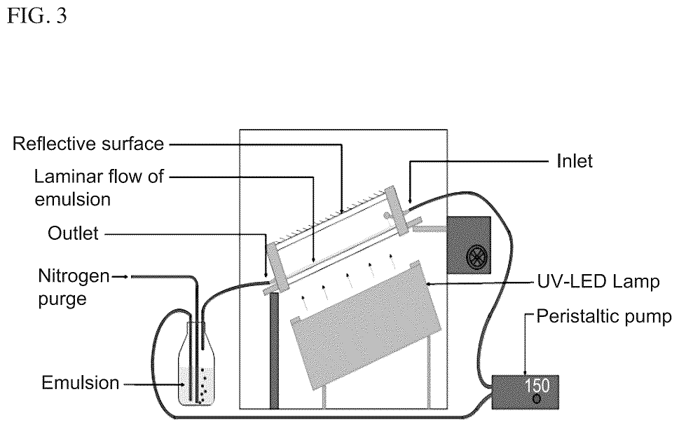

The reactor, as shown in FIG. 3, consisted of an LED panel, which emitted UV light of the wavelength 365 nm. It also included a peristaltic pump that pumped the feed to a UV reaction box through a flow distributor. The UV reaction box had a flow distributor to ensure the laminar flow of the feed on the bottom plate made of quartz. The light from the UV-LED panel was incident on this bottom plate. The upper plate of the UV reactor box was a reflecting surface to ensure maximum exposure of UV light to the feed. The feed was purged with nitrogen gas throughout the experiment, and the feed was under continuous recirculation. The flow of the feed was maintained to about 300 ml/min. The setup also included an exhaust fan to ensure that there was no overheating. All the inner surfaces of the reactor were lined with aluminum sheets to contain the scattered light.

### Methods

### 1) Emulsification

The emulsification process of aqueous and organic phases was carried out using the proportions stated in Table 1. A Silverston L5M, high shear mixer with a fine screen was used for the emulsification process. The rate of mixing was set to 4000 rpm for all samples unless otherwise mentioned.

TABLE 1
Chemical recipe of ingredients for emulsion preparation.

| | | Weight |
|---|---|---|
| | Compound | (g) |
| Aqueous Phase | Deionized water | 400 |
| | Polyvinyl alcohol (PVA) | 4 |
| | Sodium dodecyl sulphate (SDS) | 0.2 |
| | Methyl Methacrylate (MMA) | 24.71 |
| | Ethylene glycol dimethacryalte (EGDM) | 8.43 |
| Organic Phase | Pentaerythritol triacrylate (PETA) | 2.05 |
| | Puretemp ®PT20 (PCM) | 35.73 |
| | IRGACURE 819 (photoinitiator) | 1.03 |

### 2) Photo-Induced Polymerization

The encapsulation was carried out in the UV-LED reactor. The flow rate of 300 ml/min was chosen to ensure sufficient exposure time and flow as a thin laminar film. High power (450 W) and low power (150 W) of UV light incidence were tested as parameters for different treatment times in this experiment. The treatment times of 10 min, 20 min and 30 min were selected for each power. Additional treatments of 1 min, 2 min and 5 min were also conducted for the UV power of 450 W. Each sample was then centrifuged at 10000 rpm for min. The cake formed was separated to procure the microcapsules. The microcapsules were washed with n-hexane to remove the unreacted monomer and unencapsulated PCM. Finally, the washed microcapsules were dried in a hot air oven at 50° C. overnight before further analysis. Results showed high quality microcapsules. Lower UV intensity showed a reasonable microencapsulation. However, the use of treatment intensity of 450 W (one lamp) did not only give high-quality PCM microcapsules with about ≈70% PCM content, but it was possible to reduce treatment time to as low as 1.0 min.

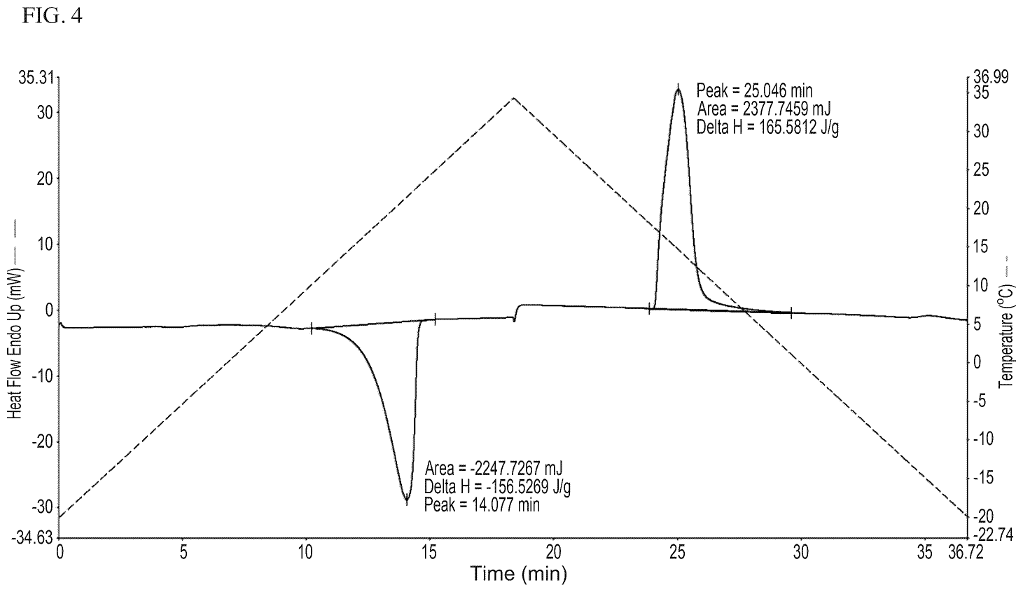

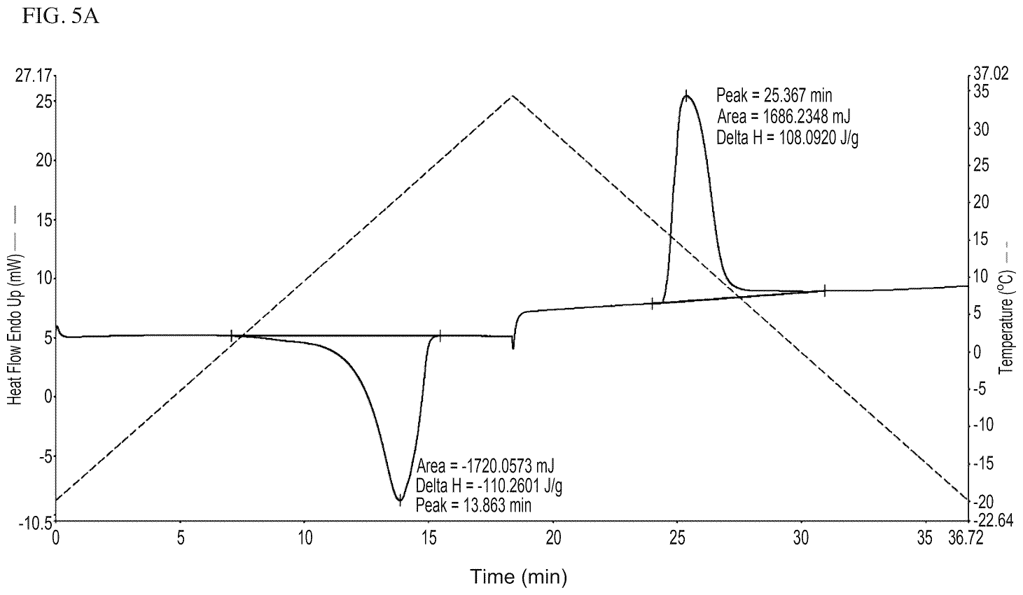

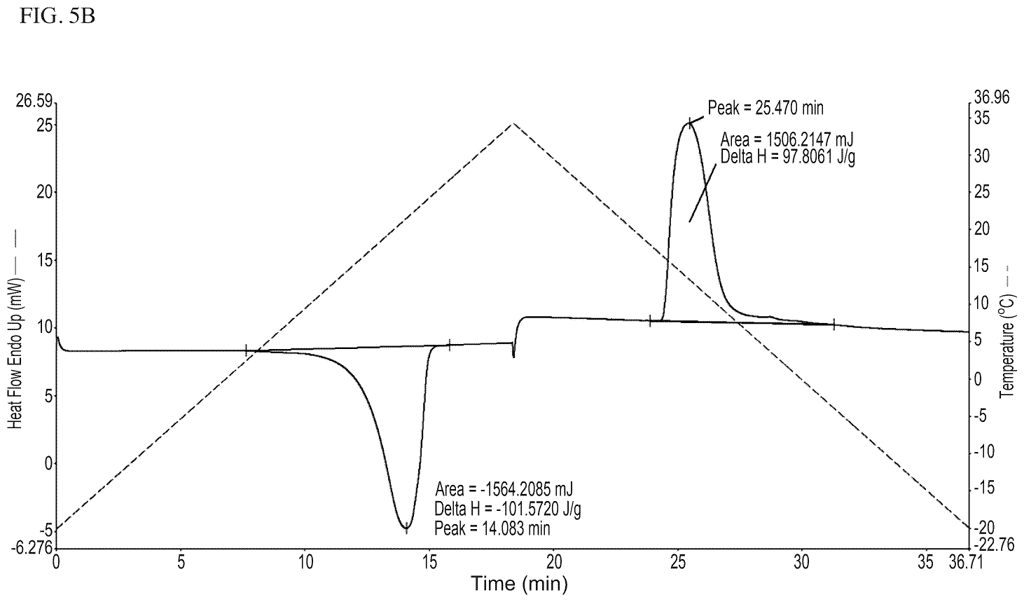

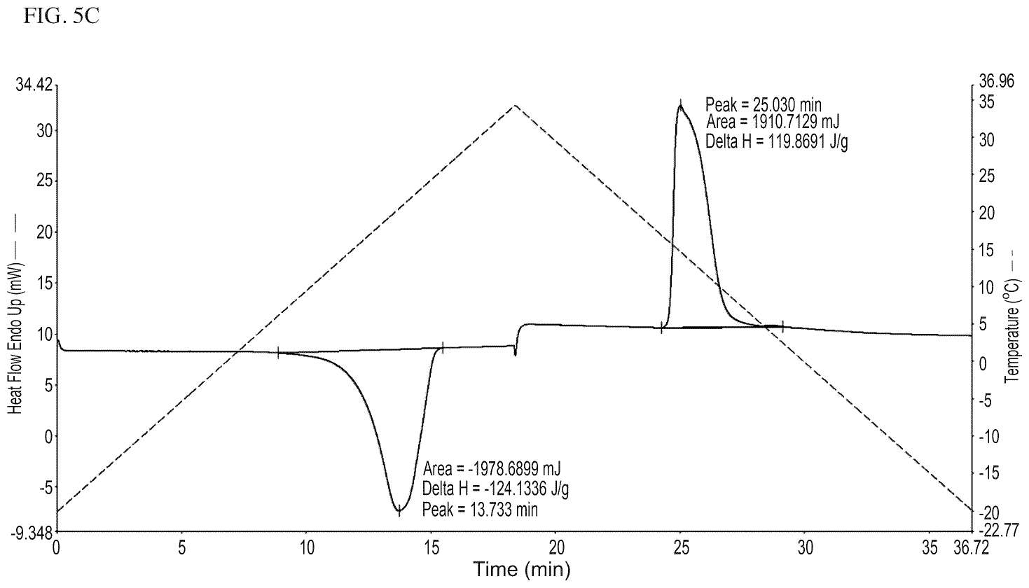

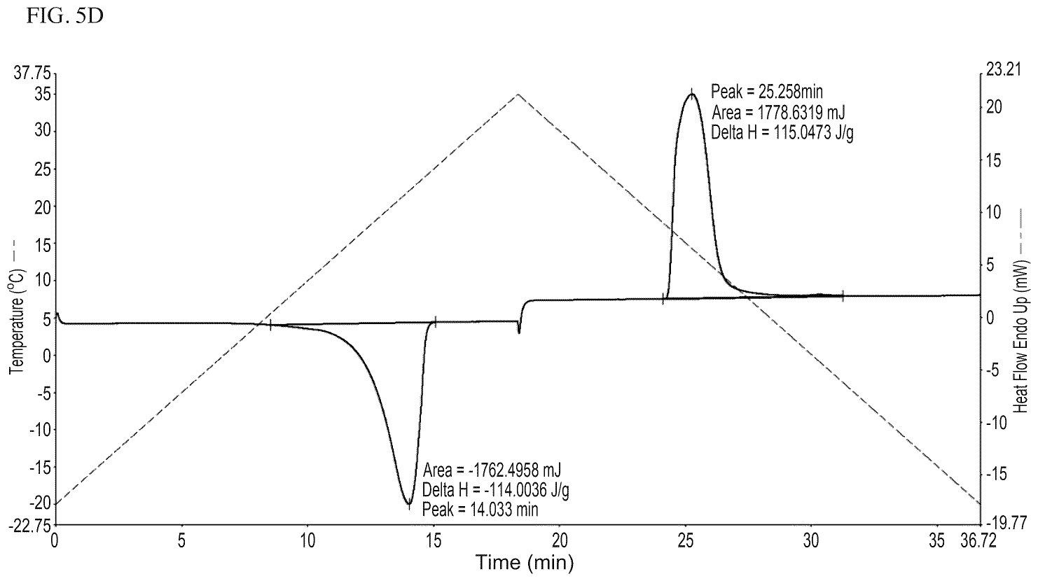

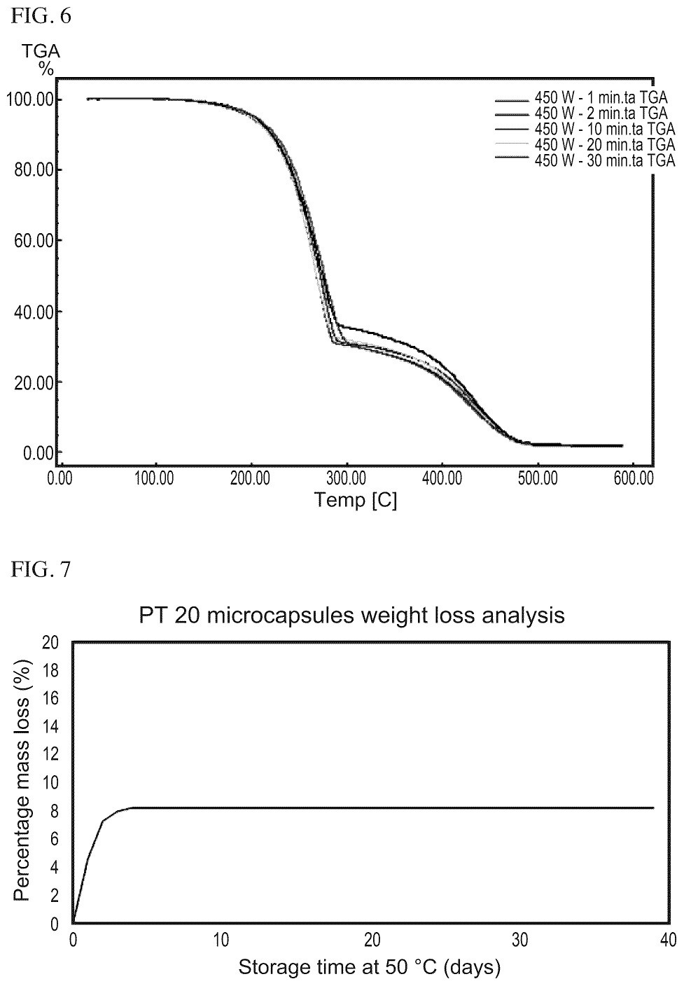

FIG. 4 show the DSC diagram of pure PT 20 PCM used in the encapsulation showing 165 J/g latent heat of melting. The corresponding latent heat of melting for different doses of UV (different treatment times) are shown in FIG. 5, showing latent heat of melting as high as 120 J/g, even for very short treatment time of 1 min only. The TGA of the microcapsules shown in FIG. 6 indicate the quality of the capsules for the 1 min is as good as those produced with longer treatment time. FIG. 7 is the strong evidence of the quality of the microcapsules since they showed stable mass when left in oven at 50° C. for an extended period. The initial loss is not PCM loss but rather the external solvent and water at the surface of the capsules.

### Example 3: Effect of Radiation Intensity on Encapsulation Efficiency for 2 Minute Reaction Time at a Wavelength of 365 nm

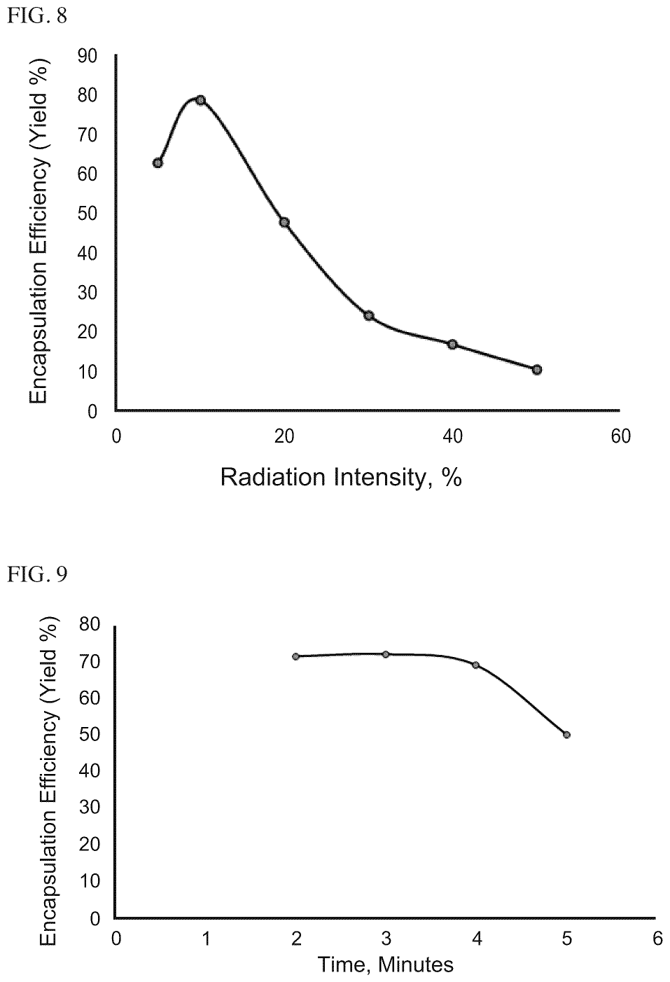

The same experimental procedures in Example 1 was used in Example 3. The encapsulation efficiency (total yield) were determined at different constant radiation intensities as shown in FIG. 8.

It is clear that there is an optimum radiation intensity where the encapsulation efficiency can be optimized. This indicates that not only matching the wavelength that the photo initiator will absorb is required but also, the intensity of radiation, which can be absorbed by the required photo reaction, should match. The peak represents the optimum radiation intensity. Below or above this optimum intensity, the encapsulation efficiency is significantly decreased.

### Example 4: Effect of Irradiation Time on Encapsulation Efficiency at a Radiation Intensity of 1.2 W/cm² (10% of the Maximum) and a Wavelength of 365 nm

The same experimental procedures in Example 1 was used in Example 4. The encapsulation efficiency (total yield %) was determined at different irradiation times at a wavelength of 365 nm and a radiation intensity of 1.2 W/cm² as shown in FIG. 9.

Increasing the time more than the required time for the encapsulation reaction may ruin the capsules. This is because the radiation will not be absorbed and its action might be to ruin or reverse the encapsulation reaction.

### Example 5: Effect of Irradiation Time on Encapsulation Efficiency at a High Radiation Intensity of 12 W/cm² (Maximum Light Intensity of the LED Lamps) and a Wavelength of 365 nm

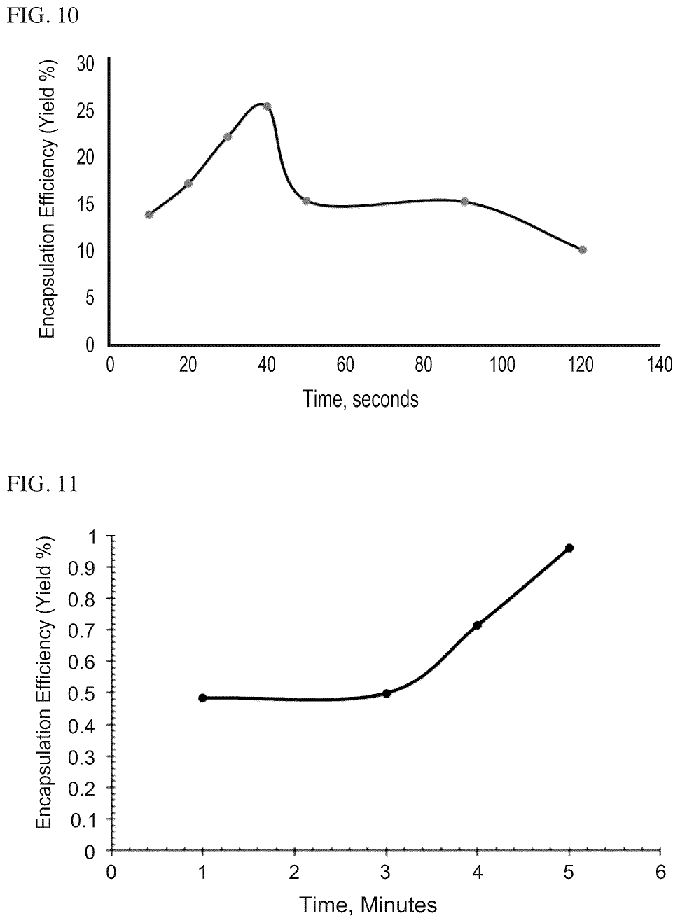

The same experimental procedures in Example 1 was used in Example 5. The encapsulation efficiency (total yield %) was determined at different irradiation times at a wavelength of 365 nm and a radiation intensity of 12 W/cm² (maximum light intensity of the LED lamps) as shown in FIG. 10.

It is clear that the encapsulation efficiency increases in the first 40 seconds, and then the high light intensity starts to have more ruin action on the encapsulation process with time due to the thermal influence of strong light on the polymerized part. The encapsulation efficiency (total yield %) at the maximum light intensity of the LED lamps does not exceed 25%. Waxy appearance of the capsules indicates that the polymeric capsule around the PCM is not completely formed, and the PCM is leaked out. This example is only to show the negative influence of the light intensities particularly on the early stage of encapsulation.

### Example 6: Effect of Decreasing or Increasing the Light Intensity on the Encapsulation Efficiency at a Wavelength of 365 nm, and Total 6 Minutes Irradiation Time

The same experimental procedures in Example 1 was used in Example 6. The encapsulation efficiency (total yield %) was determined at different irradiation times and different intensities at a wavelength of 365 nm as shown in Table 2.

TABLE 2
Encapsulation efficiency at decreasing and increasing light intensities
for total irradiation of 6 minutes, which is divided into two stages.

| Light Intensity Time | Encapsulation |
|---|---|
| 100% & 10% 3 minutes each | 34.7 |
| 10% & 100% 3 minutes each | 87.5 |
| 100% & 10% 4 minutes & 2 minutes | 25.5 |
| 10% & 100% 4 minutes & 2 minutes | 78.2 |

The encapsulation efficiency is low when it was started with a high light intensity (100%). Further reduction occurred when the 100% intensity was used for a longer time (4 minutes instead of 3 minutes). Switching to a lower light intensity (10%) did not improve efficiency. Starting with low light intensity was useful in increasing the encapsulation efficiency. However, switching from 100% to 10% intensity after 3 minutes will lead to a higher efficiency than switching after 4 minutes. This means that at each stage a certain light intensity has an optimum period of time. Using a long time in any of the stages might have a negative influence on the encapsulation process. This is similar to thermal microencapsulation, since a good capsule can be produced when polymerization temperature is kept low initially to give sufficient time for the proper shell to form. Hence in the UV-LED microencapsulation, it is necessary to start with a low radiation intensity and then increase it to a high level.

### Example 7: Effect of Irradiation Time on Encapsulation Efficiency at a Radiation Intensity of 0.6 W/cm² (5% of the Maximum) and a Wavelength of 365 nm

The same experimental procedures in Example 1 was used in Example 7. The encapsulation efficiency (total yield %) was determined at different irradiation times at a wavelength of 365 nm and a radiation intensity of 0.6 W/cm² as shown in FIG. 11.

As shown in FIG. 11, increasing the irradiation time to 5 minutes at a radiation intensity of 0.6 W/cm², it was possible to obtain encapsulation efficiency (total yield) of 95.9% and 100% PCM efficiency. The heat of fusion is 112.51 J/g when Rubitherm® RT21 as a PCM material was used. A 5 minute reaction time was needed to achieve 91.8% monomer conversion. These results are outstanding compared to all the results found in literature and described in the background herein.

### Example 8: Eliminate the Undesirable Wavelengths in the Irradiating Radiations is Essential to Avoid Unnecessary Heating and to Prevent Undesirable Side Photoreactions

Photo-encapsulation of RT21 as a phase change material (PCM) with photo-curing a mixture of MethylMetacrylate monomer (MMA) and Ethylene Glycol DiMethacryalte (EGDM) as a crosslinking agent was studied by using Cary 60 UV-Vis Spectrophotometer (made by Agilent). Scanning the absorbance of radiation by the stabilized emulsion of the organic phase dispersed in water, components with and without photoinitiator were conducted by using a range of radiation wavelength between 200 nm and 800 nm (1.5 nm fixed spectral bandwidth) and absorbance between zero and 10. The photoinitiator (PI) was bis-acylphosphine oxide (BAPO).

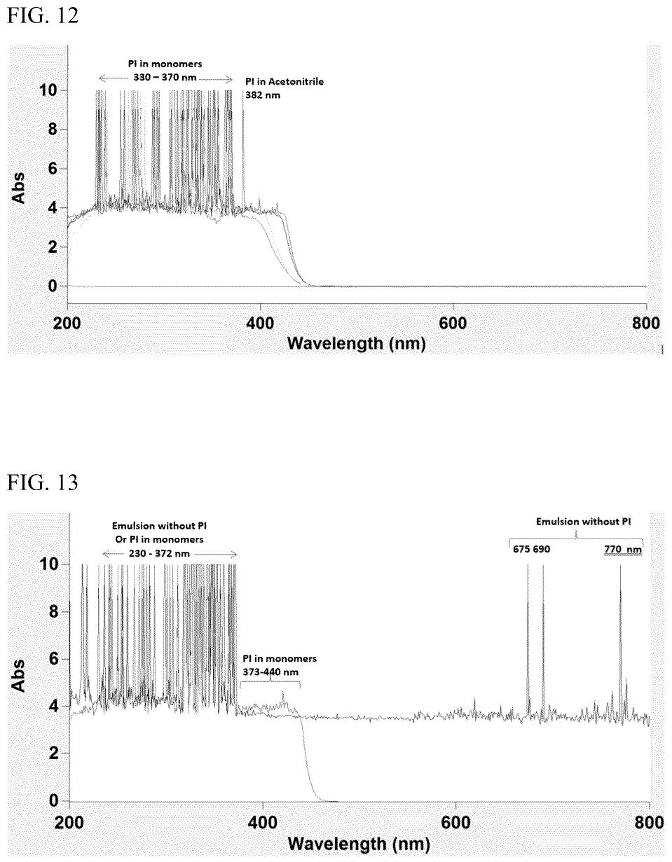

FIG. 12 shows the absorption spectra of the photoinitiator (PI) in different monomers and crosslinking agents in acetonitrile. The absorbance of the photoinitiator (PI) was between 330 and 370 nm wavelengths. PI in acetonitrile has also a peak at 382 nm wavelength. The absorbance of PI for radiations starts with decreasing the wavelength below 430-450 nm, and at a wavelength below 400 nm, absorbance becomes above 3.

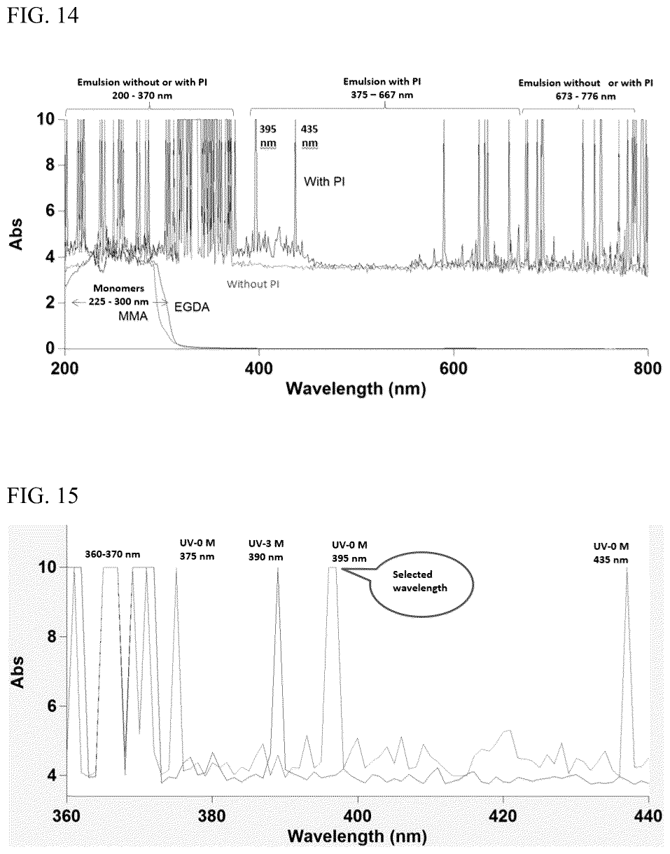

FIG. 14 shows the absorption spectra of the microencapsulation emulsion with and without photoinitiator (PI) compared to the spectra of the monomers and crosslinking agents. The absorbance of the monomers and cross-linking agents is between 225 nm and 300 nm. Excluding the ranges where there is absorption of radiation by the emulsion without PI, the absorbance of the emulsion with photoinitiator is between 375 nm and 667 nm. In this range, the emulsion with PI shows peaks at 395 nm, 435 nm, 590 nm, 626 nm, 633 nm, small at 635 nm, 657 nm and small at 667 nm.

FIG. 14 suggests that using radiation at a wavelength 395 nm or 435 nm, it is possible to minimize the radiation absorption by emulsion and maximize the radiation absorption by the photoinitiator.

To investigate the selection of the best radiation-wavelength for the photo-microencapsulation of the system, we compared the absorbance spectra of emulsion with PI before and after radiation curing for 3 minutes as shown in FIG. 15. It is clear that the absorption peak shifts to a lower wavelength by 5 nm after curing radiation for 3 minutes. The absorbance peak at 395 nm shifts to 390 nm and at 375 nm shifts to 370 nm. Also, the absorbance peak at 435 nm disappears after curing for 3 minutes.

FIGS. 12, 13, 14, and 15 suggest that the absorbance peak of the bis-acylphosphine oxide (BAPO) photoinitiator is affected by the surrounding medium. In the aqueous emulsion, the photo-absorption peaks of the photoinitiator (PI) occurs at different wavelengths compared to the absorbance peaks of PI in organic phase. Although curing occurs in emulsion micro/nano encapsulation at wavelength of 365 nm, it is possible to improve the photo-encapsulation process by using a radiation at a wavelength of 395 nm. At this wavelength, a high radiation absorption by the components existing in the emulsion can be excluded, and so the photoinitiator is mainly irradiated. This can reduce the side photo-reactions and local heating resulting from the radiation absorption that has no contribution in the photo-encapsulation by the selective radicals of the photoinitiator.

### LIST OF TABLES

Table 1: Chemical recipe of ingredients for emulsion preparation
Table 2. Encapsulation efficiency at decreasing and increasing light intensities for total irradiation of 6 minutes, which is divided into two stages.

### REFERENCES

[1] Maninder Singh, J. S. Dua, Muse Menra, Mansi Soni and D. N. Prasad; Microencapsulation And Its Various Aspects: A Review, International Journal of Advanced Research (2016), Volume 4, Issue 6, 2094-2108.
[2] Mishra, Munmaya (Editor), Handbook of Encapsulation and Controlled Release, book Published by CRC Pr I Llc (2015), ISBN 10:1482232324 ISBN 13:9781482232325
[3] Giamberini (2015) Microencapsulation: Innovative Applications; Ed. by Giamberini, Marta/Fernandez Prieto, Susana/Tylkowski, Bartosz; November 2015, ISBN 978-3-11-033199-8.
[4] Miaomiao Hu, Jintang Guo, Yongjin Yu, Lei Cao and Yang Xu; Research Advances of Microencapsulation and Its Prospects in the Petroleum Industry; Materials 2017, 10(4), 369; doi:10.3390/ma10040369
[5] Silva, Pablo Teixeira da, Fries, Leadir Lucy Martins, Menezes, Cristiano Ragagnin de, Holkem, Augusto Tasch, Schwan, Carla Luisa, Wigmann, Évelin Francine, Bastos, Juliana de Oliveira, & Silva, Cristiane de Bona da. (2014). Microencapsulation: concepts, mechanisms, methods and some applications in food technology. Ciência Rural, 44(7), 1304-1311. https://dx.doi.org/10.1590/0103-8478cr20130971
[6] Poshadri A. and Aparna Kuna. Microencapsulation Technology: a review, J.Res. ANGRAU, 2010, 38(1)86-102.
[7] A. Jamekhorshid, S. M. Sadrameli, and M. Farid, A review of microencapsulation methods of phase change materials (PCMs) as a thermal energy storage (TES) medium, Renewable and Sustainable Energy Reviews 31 (2014) 531-542].
[8] Fang Z and Bhandari B (2010) Encapsulation of polyphenols—a review. Trends Food Sci. Technol., 21:510-523. <http://dx.doi.org/10.1016%2Fj.tifs.2010.08.003>
[9] Methavee Peanparkdee, Satoshi Iwamoto, Ryo Yamauchi, Microencapsulation: A Review Of Applications In The Food And Pharmaceutical Industries, Reviews in Agricultural Science, 4:56-65, 2016. doi: 10.7831/ras.4.56.

[10]

http://bioencapsulation.net/220_newsletter/BI_2013_03/Bioencap_innov_March_2013.pdf.
[11] Stephen M. HSU, and Fei ZHAO; Microencapsulation of Chemical Additives; US 2017/0073610 A1; Mar. 16, 2017.
[12] Kalishek, Robert J. and Hayford, Donald E., Process for microencapsulation, U.S. Pat. No. 5,164,126 Nov. 17, 1992.
[13] Al-Shannaq, R., Farid, M., Al-Muhtaseb, S. & Kurdi, J., “Emulsion stability and cross-linking of PMMA microcapsules containing phase change materials.” Solar Energy Materials and Solar Cells, 132 (2015) 311-318.
[14] Scharp, David and Mccord, Roy, Encapsulation Methods and Compositions, US 2017/0252304 A1, Sep. 7, 2017.
[15] https://www.abbexa.com/4-arm-peg-acrylate
[16] http://www.borica.com/Hymer/hymer_newsletter.aspx
[17] Farid, M., Al-Shannaq, R., Al-Muhtaseb, S. & Kurdi, J., Method for Low temperature microencapsulation of phase change materials, US 2018/0223146 A1, Aug. 9, 2018.
[18] Céline Dietlin, Stéphane Schweizer, Pu Xiao, Jing Zhang, Fabrice Morlet-Savary, Bernadette Graff, Jean-Pierre Fouassier and Jacques Lalevée; Photopolymerization upon LEDs: new photoinitiating systems and strategies; Polymer Chemistry 6(21), June 2015.
[19] Kites, Andrew and Lawson, Douglas, Controllable curing systems and methods including an led source, US 20110119949, May 26, 2011.
[20] Niels Jorgen Madsen; David George Anderson; Bo Rud Nielsen; Novel Polymeric Photoinitiators and Photoinitiator Monomers; US 20150166716 A1, Jun. 18, 2015.
[21] FlorentJasinski, Per B. Zetterlund, André M. Braun, Abraham Chemtob; Photopolymerization in dispersed systems, Progress in Polymer Science, Volume 84, September 2018, Pages 47-88.

We claim:

1. A cost-eco-efficient method for rapid photochemical micro/nano-encapsulation of an active material with a polymeric shell at ambient or cold temperatures to produce high-quality shell-core micro/nano-capsules wherein the method comprises the following steps:
a) Selecting photo-curable resins which have a close hydrophilicity to the active material and are suitable for forming a solid shell with desirable characteristics;
b) Selecting at least one rapid dissociation triplet Norrish-Type I free-radical photo-initiator with a complete photobleaching ability and that are soluble in the curable resins;
c) Dispersing the active material, the photo-curable resins, and photo-initiator(s) in an immiscible light-transparent continuous liquid phase using a stabilizer-emulsifier without surfactant
d) Using a compact effective-space photoreactor, which is equipped properly with a stirrer and LED lamps, to photo-encapsulate the resins with the photo-initiator inside the dispersed phase efficiently and rapidly, at ambient or below ambient temperatures, to afford a curable material; and
e) Adding to the curable materials a second mono-functional monomer, a di, tri, poly, functional monomers, and/or additives selected from the group consisting of oxygen-containing (ethers) nitrogen-containing (amines, amino-monomers), sulfur-containing (thiols), redox initiators, or other commercial additives to improve the encapsulation process and/or to obtain the desired characteristics of the shell solid layer in the capsules.

2. The method of claim 1, wherein a high-shear homogenizer and/or sonication device is used in step (c) to obtain the desired size of the dispersed phase.

3. The method of claim 1, wherein the photoreactor in step (d) is a transparent square or cylindrical tube with a diameter between 3 to 4 cm wherein the tube is oriented vertically and located below the emulsion tank, and/or the flow of emulsion occurs by gravity falling from a emulsion tank through the tube and down to a product tank.

4. The method of claim 3, wherein the photoreactor is equipped with a least one LED lamp, which touches the tube perpendicularly to achieve the shortest light path length with no air gap to avoid ray diffraction.

5. The method of claim 4, wherein the photoreactor is equipped with more than one LED lamp.

6. The method of claim 3, wherein the photoreactor is equipped with a laminar low shear stirrer.

7. The method of claim 1, wherein in step (d) the photo-initiator is irradiated by an LED lamp or/and Visible LED lamp having an appropriate narrow or single wavelength at an adjustable intensity for ranges of multi-interval times.

8. The method of claim 7, wherein the photo-initiator is irradiated by the UV or/and Visible LED lamp at optimum wavelengths, light intensities and reactions times for complete conversion of all curable-material and full encapsulation of the active material.

9. The method of claim 7, wherein the selected radiation is a narrow range of wavelength in which the undesirable wavelengths that causes unnecessarily heating are eliminated.

10. The method of claim 1, wherein the method affords complete up to 100% active material encapsulation, yield above 95%, and a monomers conversion is above 90% at a photoencapsulation temperature of 22° C. (ambient T) for 5 minutes.

11. The method of claim 1, wherein the UV intensity depends on the formula of curable materials.

12. The method of claim 1, wherein the selected radiation is a narrow range of wavelength in which the undesirable wavelengths that cause unnecessarily heating are eliminated.
